SEQUENCE LISTING (8 sequences)

SEQ ID NO: 1 - DNA, 933 bp, Unknown
  no basis in the application as filed for the name of the microorganism
atgagcaagccaatcgagatcgagattcgcagagcgcccgtgctgggaagcagcatggcttaccgcgagacgggtgcgcaggatgcgccggtcgtgctgttcctgcacggcaacccgacctcgtcgcacatctggcgcaacatcctgccgttggtgtcaccggtcgcgcattgcattgcgcccgatctcatcggcttcggccaatccggtaagcctgacatcgcctaccgcttcttcgaccatgtccgctatctcgatgcgttcatcgaacagcgcggcgtcacatcggcctatctcgtcgcgcaggactggggcacggcgctcgcatttcatctcgccgcgcgccggccggatttcgtacgcggattagccttcatggaattcatccgcccgatgccgacctggcaggatttccaccataccgaggtcgcggaggagcaagatcatgccgaggcggcgagggcggtctttcgcaagttcaggacgccgggcgagggtgaggccatgatcctcgaggcgaatgcgttcgtcgagcgcgttctgcccggcggaatcgtccgcaagctcggcgacgaagaaatggcgccctatcgcacgccgttcccgacgcccgagagtcgccgccccgttcttgcgtttccccgcgagctgccgatcgcaggtgagcctgccgatgtctatgaggcgctccaatccgcccatgcggcgctggccgcatcttcctatccgaaactgctgttcacgggcgaaccgggcgcgctcgtctcgccggaatttgccgagcggtttgcggcctcgctgacgcgttgcgcgttgatccggctcggcgcgggattgcactatctgcaggaggaccacgctgacgcaatcggccgatcggtggccggctggatcgccggcatcgaagcggtgcgtccgcagctcgccgcgtga

SEQ ID NO: 2 - DNA, 891 bp, sphingomonas paucimobilis
atgagcctcggcgcaaagccatttggcgagaagaaattcattgagatcaagggccggcgcatggcctatatcgatgaagggaccggcgatccgatcctcttccagcacggcaatccgacgtcgtcctatctgtggcgcaatatcatgccgcattgcgccgggctgggacggctgatcgcctgtgacctgatcggcatgggcgattcggacaagctcgatccgtcggggcccgagcgttatgcctatgccgagcatcgtgactatctcgacgcgctgtgggaggcgctcgatctcggggacagggttgttctggtcgtgcatgactgggggtccgccctcggcttcgactgggcccgccgccaccgcgagcgtgtacaggggattgcctatatggaagcgatcgccatgccgatcgaatgggcggattttcccgaacaggatcgcgatctgtttcaggcctttcgctcgcaggcgggcgaagaattggtgttgcaggacaatgtttttgtcgaacaagttctccccggattgatcctgcgccccttaagcgaagcggagatggccgcctatcgcgagcccttcctcgccgccggcgaagcccgtcgaccgaccctgtcttggcctcgccaaatcccgatcgcaggcaccccggccgacgtggtcgcgatcgcccgggactatgccggctggctcagcgaaagcccgattccgaaactcttcatcaacgccgagccgggagccctgaccacgggccgaatgcgcgacttctgccgcacatggccaaaccagaccgaaatcacggtcgcaggcgcccatttcatccaggaggacagtccggacgagattggcgcggcgattgcggcgtttgtccggcgattgcgcccagcataa

SEQ ID NO: 3 - DNA, 882 bp, Rhodococcus rhodochrous
atgtcagaaatcggtacaggcttccccttcgacccccattatgtggaagtcctgggcgagcgtatgcactacgtcgatgttggaccgcgggatggcacgcctgtgctgttcctgcacggtaacccgacctcgtcctacctgtggcgcaacatcatcccgcatgtagcaccgagtcatcggtgcattgctccagacctgatcgggatgggaaaatcggacaaaccagacctcgattatttcttcgacgaccacgtccgctacctcgatgccttcatcgaagccttgggtttggaagaggtcgtcctggtcatccacgactggggctcagctctcggattccactgggccaagcgcaatccggaacgggtcaaaggtattgcatgtatggaattcatccggcctatcccgacgtgggacgaatggccggaattcgcccgtgagaccttccaggccttccggaccgccgacgtcggccgagagttgatcatcgatcagaacgctttcatcgagggtgcgctcccgaaatgcgtcgtccgtccgcttacggaggtcgagatggaccactatcgcgagcccttcctcaagcctgttgaccgagagccactgtggcgattccccaacgagctgcccatcgccggtgagcccgcgaacatcgtcgcgctcgtcgaggcatacatgaactggctgcaccagtcacctgtcccgaagttgttgttctggggcacacccggcgtactgatccccccggccgaagccgcgagacttgccgaaagcctccccaactgcaagacagtggacatcggcccgggattgcactacctccaggaagacaacccggaccttatcggcagtgagatcgcgcgctggctccccgcactctag

SEQ ID NO: 4 - DNA, 903 bp, Unknown
  no basis in the application as filed for the name of the microorganism
atgacagcattcggcgtcgagccctacgggcagccgaagtacctagaaatcgccgggaagcgcatggcgtatatcgacgaaggcaagggtgacgccatcgtctttcagcacggcaaccccacgtcgtcttacttgtggcgcaacatcatgccgcacttggaagggctgggccggctggtggcctgcgatctgatcgggatgggcgcgtcggacaagctcagcccatcgggacccgaccgctatagctatggcgagcaacgagactttttgttcgcgctctgggatgcgctcgacctcggcgaccacgtggtactggtgctgcacgactggggctcggcgctcggcttcgactgggctaaccagcatcgcgaccgagtgcaggggatcgcgttcatggaagcgatcgtcaccccgatgacgtgggcggactggccgccggccgtgcggggtgtgttccagggtttccgatcgcctcaaggcgagccaatggcgttggagcacaacatctttgtcgaacgggtgctgcccggggcgatcctgcgacagctcagcgacgaggaaatgaaccactatcggcggccattcgtgaacggcggcgaggaccgtcgccccacgttgtcgtggccacgaaaccttccaatcgacggtgagcccgccgaggtcgtcgcgttggtcaacgagtaccggagctggctcgaggaaaccgacatgccgaaactgttcatcaacgccgagcccggcgcgatcatcaccggccgcatccgtgactatgtcaggagctggcccaaccagaccgaaatcacagtgcccggcgtgcatttcgttcaggaggacagcccagaggaaatcggtgcggccatagcacagttcgtccggcagctccggtcggcggccggcgtctga

SEQ ID NO: 5 - DNA, 903 bp, unknown
  no basis in the application as filed for the name of the microorganism
atggatgtcctacgcaccccagactcccggttcgaacacctggtgggctacccgtttgcaccgcactatgtcgatgtgacggccggcgacacccagccgttgcgaatgcactacgtcgacgagggcccgggcgacggtccgccgatcgtcttgctgcacggcgagcccacctggagttatctgtaccgaaccatgattccgccgctctccgccgccgggcaccgtgtgctcgcgcccgacctgatcggcttcggccgctccgacaagccgactcgcatcgaggactacacctacctgcggcacgtcgagtgggtgacgtcctggttcgagaatctcgacctgcacgacgttacgctcttcgtgcaggactgggggtcattgatcggtctgcgcatcgctgccgagcacggtgaccggatcgcgcggctggtggtcgccaacgggtttctccccgccgcgcaggggcgcaccccactccccttctacgtgtggcgggcgtttgcgcgctattctccggtgcttcccgctggccgtctggtgaacttcggcaccgtccacagggttcccgccggggtccgagccggctacgatgcacctttccccgacaaaacgtatcaagccggcgcccgggcgttcccacggttggtgccgacctcacccgacgatccggcggtaccggccaaccgcgcggcatgggaagccctgggccggtgggacaaaccgttccttgccatcttcggttatcgcgacccgatactcgggcaagcggacggtccgctgatcaagcacattcccggcgcggcgggtcagccgcacgcccgcatcaaggccagccacttcatccaggaggacagcggaaccgaactcgccgaacgcatgctctcctggcagcaggcaacgtaa

SEQ ID NO: 6 - DNA, 861 bp, unknown
  no basis in the application as filed for the name of the microorganism
atgagcatcgatttcacgccggatccgcagctgtacccgttcgagtcgcgctggttcgacagctcgcgtggacgcatccactacgtcgacgagggcacgggtccgccgatcctgttgtgtcacggcaacccgacgtggagtttcctgtatcgggacatcatcgtcgcactgcgggaccgtttccgttgtgtggctccggattatctgggtttcgggttatcggagcgtccctcgggattcgggtaccagatcgacgagcacgcgcgggtgatcggcgaattcgtcgatcacctgggcctggaccgctacctgagcatgggtcaggactggggtggcccgatcagcatggcggtcgctgtcgagcgtgccgaccgggtccgcggcgtcgtgttgggcaacacgtggttctggccggcggacacgctggcgatgaaggccttcagcagggtgatgtccagcccgccagtgcagtacgcgatcttacggcgcaacttctttgtcgagcgcttgatacccgcgggaaccgagcaccggccgagtagcgcggtgatggcgcactaccgggcggtgcagcccaacgccgcggcacgccgaggcgtagccgagatgcccaaacagatcctggccgcccgtcccctgctggcacggctcgcccgggaggtgccagccacgctgggcaccaagcccaccctgttgatttgggggatgaaggatgtcgcattcaggccgaaaacgattatccccagactgagtgcgacatttcccgaccacgtcctggtggagctgcccaacgccaaacacttcatccaggaggacgcccccgaccggatcgccgccgcgatcattgagcgcttcggctga

SEQ ID NO: 7 - DNA, 987 bp, unknown
  no basis in the application as filed for the name of the microorganism
ttgagttgccgcctctcgtcaaatcgccgcggatcgtcgaaactagccgccatgacgaatcttgctagcgatctgtttccccacccgtcgtcggaattgtccatcgacggtcacacgctgcgctacatcgatacggcggccagctctgacatcccgagttccgcggtcggatcctccgatggcgagccaacgtttctttgtgtgcatggcaatccgacgtggagcttttactaccggcgaatcatcgagcggtatggcaagcagcaacgagtgatcgcggtcgatcacatcggttgtggtcgcagcgacaaaccatcggaagacgaattcccgtacacgatggccgcgcatcgagacaacctgattcggttggtcgacgagttggatctgaagaacgtgatcctgatcgctcacgattggggtggtgcgattggtttgtcagccatgcatgctcgccgagaccgcttggctgggattgggttgctgaacacggctgcgttcccaccgccgtacatgcctcagcgaattgccgcgtgccggatgccggtgttgggaactcccgcagttcgcggattgaacttgttcgcacgggccgcggtcaccatggccatgtcgcgtacgaagatgaaacccgatgtcgcagcgggattgctggctccctatgacaattggaagaaccgagtcgcaatcgatcggttcgttcgcgacattcctttgaatgattcgcatcccacgatgaagactcttcggcagctggagtccgatctgccggacctggcatcgctacccatctctttgatttggggaatgaaggattggtgttttcgaccggaatgtctgcgacgtttccaatccgtttggcccgacgcggaagtcacggaactggcgacgaccggtcactatgtgatcgaagactcgcccgaagaaaccttggccgcgattgattcattgctcgcccgcgtcaaggaacgcatcggtgcggcgtga

SEQ ID NO: 8 - DNA, 906 bp, unknown
  no basis in the application as filed for the name of the microorganism
atgcatgtgctgcgaaccccggactcccgattcgaaaacctggaggactacccgttcgtggcgcattatctcgacgtcaccgcgcgcgacacccggccgcttcgcatgcactacctggacgaggggccgatcgacgggccaccgatcgtgctgctgcacggcgagcccacctggagctacctgtaccgcaccatgatcacgccgctgaccgacgccggaaaccgggtgctggcacccgacttgatcggcttcggccggtcggacaagcccagccggatcgaggactactcctaccagcggcacgtggactgggtggtctcctggttcgaacacctcaacctcagcgacgtcacgctgttcgtgcaggactggggatcattgatcgggctgcgcatcgccgccgagcagcccgaccgggtgggacggctggtggtggccaacggctttcttcccaccgcgcagcgacgcaccccgcccgccttctacgcgtggcgagccttcgcgcgctactcccccgtgctgcccgccggccgcatcgtcagcgtcgggaccgtccgccgggtttcgtccaaggtgcgtgccggctacgacgcgcccttccccgacaagacgtatcaggccggggcgcgggcatttccgcaactggtgcccacctcgccggccgatcccgcgattccggccaaccgcaaggcgtgggaagccctcggccgctgggaaaaaccgttcctggccatcttcggcgcccgcgaccccatcctcggccacgcggacagtccgctgatcaagcacattccgggcgccgcgggccaaccgcacgcccgcatcaacgccagtcacttcatccaggaggaccgcggacctgaactggccgaacgcatcctgtcctggcagcaggcgctgctctga

(19) United States
(12) Patent Application Publication
(10) Pub. No.: US 2009/0155868 A1
(43) Pub. Date: Jun. 18, 2009

(54) Method of Production of Optically Active Halohydrocarbons and Alcohols Using Hydrolytic Dehalogenation Catalysed by Haloalkanedehalogenases

(75) Inventors: Zbynek Prokop, Brno (CZ); Jiri Damborsky, Brno (CZ); Dick B. Janssen, Roden (NL); Yuji Nagata, Sendai (JP)

Correspondence Address: NOTARO & MICHALOS P.C., 100 DUTCH HILL ROAD, SUITE 110, ORANGEBURG, NY, 10962-2100, US

(73) Assignee: MASARYKOVA UNIVERZITA V BRNE, Brno (CZ)

(21) Appl. No.: 11/793,635

(22) PCT Filed: Dec. 23, 2005
(86) PCT No.: PCT/CZ2005/000099

§ 371 Date: Jun. 20, 2007

(30) Foreign Application Priority Data
Dec. 27, 2004 (CZ) PV 2004-1240

(51) Int. Cl. C12P 7/02; C12P 5/00

(52) U.S. Cl. 435/155; 435/166

(57) ABSTRACT

A method of production of optically active compounds, particularly halohydrocarbons, haloalcohols, alcohols, halopolyols and polyols using hydrolytic dehalogenation of racemic or prochiral halegenhydrocarbons by dehalohenation catalysed by haloalkane dehalogenases (EC 3.8.1.5) where at least one wild type or modified haloalkane dehalogenase is affected by at least one racemic or prochiral chlorinated, brominated or iodinated compound at the temperature ranged between +10 and +70° C. and pH value between 4.0 and 12.0, in aqueous system or in a monophasic organic solution or in a monophasic organic/aqueous solution or in organic/aqueous biphasic systems.

### FIELD OF THE INVENTION

This invention relates to method for production of optically active haloalkanes, haloalcohols, alcohols, halopolyols and polyols by hydrolytic dehalogenation catalysed by an enzyme haloalkane dehalogenase (Enzyme Commission number EC 3.8.1.5) isolated from microorganisms or altered haloalkane dehalogenases with improved substrate specificity, stereo- or regio-selectivity.

### STATE OF THE ART

Enzymes are catalysts of biological systems that determine the patterns of chemical transformations. The most striking characteristics of enzymes are their catalytic power and specificity. They are highly effective catalysts for an enormous diversity of chemical reactions because of their capacity to specifically bind a very wide range of molecules. The enzymes catalyse reactions by destabilizing substrate or by stabilizing transition state and determining which one of several potential chemical reactions actually will take place.

The manufacture of enantiomerically pure compounds has become an expanding area of the fine chemical industry. When pharmaceuticals, agrochemicals, food additives and their synthetic intermediates are marketed as single enantiomers, high enantiomeric purities, typically enantiomeric excess (e.e.)>98%, are required (enantiomeric excess is derived from the concentration of the two enantiomenrs c^{R} and c^{S}; Equation 1).

$$\begin{matrix}e . e . = \vert \frac{{C}^{R} - {C}^{S}}{{C}^{R} + {C}^{S}} \vert & ( Eq . 1 ) \\ E = \frac{{( {k}_{cat} / {k}_{m} )}_{R}}{{( {k}_{cat} / {k}_{m} )}_{S}} & ( Eq . 2 )\end{matrix}$$

Enzyme-catalyzed reactions have become popular alternatives to classical chemistry for its high selectivity and activity under mild reaction conditions, and several industrial processes using enzymes as a catalyst are already in use. Clearly, the enantioselective performance of the catalyst is the single most important factor for the success of such a process (evaluation of this property is facilitated by the use of enantiomeric ratio (E); E-values can be expressed as ratio k_{cat}/Kₘ of the rate constants k_{cat} for catalysis and the Michaelis-Menten constants Kₘ of the two enantiomers; Equation 2).

Chemical transformation of halogenated compounds is important from both the environmental and synthetic point of view. Six major pathways for enzymatic transformation of halogenated compounds have been described: (i) oxidation, (ii) reduction, (iii) dehydrohalogenation, (iv) hydratation, (v) methyl transfer and (vi) hydrolytic, glutathione-dependent and intramolecular substitution. Redox enzymes are responsible for the replacement of the halogen by a hydrogen atom and for oxidative degradation. Elimination of hydrogen halide leads to the formation of an alkene, which is further degraded by oxidation. The enzyme-catalysed formation of an epoxide from a halohydrin and the hydrolytic replacement of a halide by hydroxyl functionality take place in a stereospecific manner and are therefore of high synthetic interest [Falber, K. (2000) Biotransformations in Organic Chemistry, Springer-Verlag, Heildeberg, 450].

Haloalkane dehalogenases (EC 3.8.1.5) are enzymes able to remove halogen from halogenated aliphatic compounds by a hydrolytic replacement, forming the corresponding alcohols [Janssen, D. B., Pries, F., and Van der Ploeg, J. R. (1994) Annual Review of Microbiology 48, 163-191]. Hydrolytic dehalogenation proceeds by formal nucleophilic substitution of the halogen atom with a hydroxyl ion. The mechanism of hydrolytic dehalogenation catalysed by the haloalkane dehalogenase enzymes (EC 3.8.1.5) is shown in FIG. 1. A cofactor or a metal ion is not required for the enzymatic activity of haloalkane dehalogenases. The reaction is initiated by binding of the substrate in the active site with the halogen in the halide-binding site. The binding step is followed by a nucleophilic attack of aspartic acid (Asp) on the carbon atom to which the halogen is bound, leading to cleavage of the carbon-halogen bond and formation of alkyl-enzyme intermediate. The intermediate is subsequently hydrolysed by activated water, with histidine (His) acting as a base catalyst, with formation of enzyme-product complex. Asp or glutamic acid (Glu) keeps His in proper orientation and stabilises a positive charge that develops on histidine imidazole ring during the reaction. The final step is release of the products.

The first haloalkane dehalogenase has been isolated from the bacterium Xanthobacter autotrophicus GJ10 in 1985 [Janssen, D. B., Scheper, A., Dijkhuizen, L., and Witholt, B. (1985) Applied and Environmental Microbiology 49, 673-677; Keuning, S., Janssen, D. B., and Witholt, B. (1985) Journal of Bacteriology 163, 635-639]. Since then, a large number of haloalkane dehalogenases has been isolated from contaminated environments [Scholtz, R., Leisinger, T., Suter, F., and Cook, A. M. (1987) Journal of Bacteriology 169, 5016-5021; Yokota, T., Omori, T., and Kodama, T. (1987) Journal of Bacteriology 169, 4049-4054; Janssen, D. B., Gerritse, J., Brackman, J., Kalk, C., Jager, D., and Witholt, B. (1988) European Journal of Biochemistry 171, 67-92; Sallis, P. J., Armfield, S. J., Bull, A. T., and Hardman, D. J. (1990) Journal of General Microbiology 136, 115-120; Nagata, Y., Miyauchi, K., Damborsky, J., Manova, K., Ansorgova, A., and Takagi, M. (1997) Applied and Environmental Microbiology 63, 3707-3710; Poelarends, G. J., Wilkens, M., Larkin, M. J., van Elsas, J. D., and Janssen, D. B. (1998) Applied and Environmental Microbiology 64, 2931-2936]. More recently, hydrolytic dehalogenation activity of several species of genus Mycobacterium isolated from clinical material [Jesenska, A., Sedlacek, I., and Damborsky, J. (2000) Applied and Environmental Microbiology 66, 219-222] have been reported, and haloalkane dehalogenases have been subsequently already isolated from pathogenic bacteria [Jesenska, A., Bartos, M., Czemekova, V., Rychlik, I., Pavlik, I., and Damborsky, J. (2002) Applied and Environmental Microbiology 68, 3724-3730].

Structurally, haloalkane dehalogenases belong to the α/β-hydrolase fold superfamily [Ollis, D. L., Cheah, E., Cygler, M., Dijkstra, B., Frolow, F., Franken, S. M., Harel, M., Remington, S. J., Silman, I., Schrag, J., Sussman, J. L., Verschueren, K. H. G., and Goldman, A. (1992) Protein Engineering 5, 197-211; Nardini, M., and Dijkstra, B. W. (1999) Current Opinion in Structural Biology 9, 732-737]. Without exception, haloalkane dehalogenases contain a nucleophile elbow [Damborsky, J. (1998) Pure and Applied Chemistry 70, 1375-1383; Damborsky, J., and Koca, J. (1999) Protein Engineering 12, 989-998], which is the most conserved structural feature within the α/β-hydrolase fold. The other highly conserved region in haloalkane dehalogenases is the central β-sheet. Its strands, flanked on both sides by α-helices, form the hydrophobic core of the main domain that carries the catalytic triad Asp-His-Asp/Glu. The second domain, consisting solely of α-helices, lies like a cap on top of the main domain. Residues on the interface of the two domains form the active site. Whereas there is significant similarity in the catalytic core, the sequence and structure of the cap domain diverge considerably among different dehalogenase. The cap domain is proposed to play a prominent role in determining substrate specificity [Pries, F., Van den Wijngaard, A. J., Bos, R., Pentenga, M., and Janssen, D. B. (1994) Journal of Biological Chemistry 269, 17490-17494; Kmunicek, J., Luengo, S., Gago, F., Ortiz, A. R., Wade, R. C., and Damborsky, J. (2001) Biochemistry 40, 8905-8917].

A number of haloalkane dehalogenases from different bacteria have been biochemically characterised. A principal component analysis of activity data indicated the presence of three specificity classes within this family of enzymes [Nagata, Y., Miyauchi, K., Damborsky, J., Manova, K., Ansorgova, A., and Takagi, M. (1997) Applied and Environmental Microbiology 63, 3707-3710; Damborsky, J., and Koca, J. (1999) Protein Engineering 12, 989-998; Damborsky, J., Nyandoroh, M. G., Nemec, M., Holoubek, I., Bull, A. T., and Hardman, D. J. (1997) Biotechnology and Applied Biochemistry 26, 19-25]. Three haloalkane dehalogenases representing these different classes have been isolated and structurally characterised in atomic detail so far: the haloalkane dehalogenase DhlA from Xantobacter autotrophicus GJ10 [Keuning, S., Janssen, D. B., and Witholt, B. (1985) Journal of Bacteriology 163, 635-639; Franken, S. M., Rozeboom, H. J., Kalk, K. H., and Dijkstra, B. W. (1991) The EMBO Journal 10, 1297-1302], the haloalkane dehalogenase DhaA from Rhodococcus rhodochrous NCIMB 13064 [Kulakova, A. N., Larkin, M. J., and Kulakov, L. A. (1997) Microbiology 143, 109-115; Newman, J., Peat, T. S., Richard, R., Kan, L., Swanson, P. E., Affholter, J. A., Holmes, I. H., Schindler, J. F., Unkefer, C. J., and Terwilliger, T. C. (1999) Biochemistry 38, 16105-16114] and the haloalkane dehalogenase LinB from Sphingomonas paucimobilis UT26 [Nagata, Y., Miyauchi, K., Damborsky, J., Manova, K., Ansorgova, A., and Takagi, M. (1997) Applied and Environmental Microbiology 63, 3707-3710; Marek, J., Vevodova, J., Kuta-Smatanova, I., Nagata, Y., Svensson, L. A., Newman, J., Takagi, M., and Damborsky, J. (2000) Biochemistry 39, 14082-14086]. The size, geometry and physico-chemical properties of active sites and entrance tunnels, as well as nature and spatial arrangement of the catalytic residues (catalytic triad, primary and secondary halide-stabilizing residues [Bohac, M., Nagata, Y., Prokop, Z., Prokop, M., Monincova, M., Koca, J., Tsuda, M., and Damborsky, J. (2002) Biochemistry 41, 14272-14280] can be related to the substrate specificity, which is different for enzymes representing different classes [Damborsky, J., Rorije, E., Jesenska, A., Nagata, Y., Klopman, G., and Peijnenburg, W. J. G. M. (2001) Environmental Toxicology and Chemistry 20, 2681-2689].

Several patent applications concern to dehalogenation methods using dehalogenase enzymes. For instance, the application WO 98/36080 A1 relates to dehalogenases capable of converting the halogenated aliphatic compounds to vicinal halohydrines and DNA sequences encoding polypeptides of enzymes as well as to DNA sequences and the methods of producing the enzymes by placing the expression constructs into host cells. The patent document WO 01/46476 A1 relates to methods of dehalogenation of alkylhalogenes catalyzed by altered hydrolase enzymes under formation of stereoselective or stereospecific reaction products as alcohols, polyols and epoxides; it includes also method of providing altered nucleic acids that encode altered dehalogenase or other hydrolase enzymes. The patent document WO 02/068583 A2 relates to haloalkane dehalogenases and to polynucleotides encoding the haloalkane dehalogenases. In addition, methods of designing new dehalogenases and method of use thereof are also provided. The dehalogenases have increased activity and stability at increased pH and temperature.

Although several patent applications relate to enzymatically catalysed dehalogenation, there have been no report that the specific family of hydrolytic enzymes, haloalkane dehalogenases (EC 3.8.1.5), shows sufficient enantioselectivity or regioselectivity for large-scale production of optically active alcohols. In 2001, Pieters and co-workers [Pieters, R. J., Spelberg, J. H. L., Kellogg, R. M., and Janssen, D. B. (2001) Tetrahedron Letters 42, 469-471] have investigated chiral recognition of haloalkane dehalogenases DhlA and DhaA. The magnitude of the chiral recognition was low; a maximum E-value of 9 could be reached after some structural optimization of the substrate. In the beginning of 2004, twenty years after discovery of the first haloalkane dehalogenase, the development of enantioselective dehalogenases for use in industrial biocatalysis was defined as one of the major challenges of the field [Janssen, D. B. (2004) Current Opinion in Chemical Biology 8, 150-159].

### SEQUENCE LISTING

```
(1) GENERAL INFORMATION:
    (iii) NUMBER OF SEQUENCES: 8
(2) INFORMATION FOR SEQ ID NO: 1:
        (i) SEQUENCE CHARACTERISTICS:
                (A) LENGTH: 933 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 1 (DbjA)
                10 20 30 40 50 60
....|....|....|....|....|....|....|....|....|....|....|....|
atgagcaagccaatcgagatcgagattcgcagagcgcccgtgctgggaagcagcatggct
MetSerLysProIleGluIleGluIleArgArgAlaProValLeuGlySerSerMetAla
                  70 80 90 100 110 120
....|....|....|....|....|....|....|....|....|....|....|....|
taccgcgagacgggtgcgcaggatgcgccggtcgtgctgttcctgcacggcaacccgacc
TyrArgGluThrGlyAlaGlnAspAlaProValValLeuPheLeuHisGlyAsnProThr
                130 140 150 160 170 180
....|....|....|....|....|....|....|....|....|....|....|....|
tcgtcgcacatctggcgcaacatcctgccgttggtgtcaccggtcgcgcattgcattgcg
SerSerHisIleTrpArgAsnIleLeuProLeuValSerProValAlaHisCysIleAla
                190 200 210 220 230 240
....|....|....|....|....|....|....|....|....|....|....|....|
cccgatctcatcggcttcggccaatccggtaagcctgacatcgcctaccgcttcttcgac
ProAspLeuIleGlyPheGlyGlnSerGlyLysProAspIleAlaTyrArgPhePheAsp
                250 260 270 280 290 300
....|....|....|....|....|....|....|....|....|....|....|....|
catgtccgctatctcgatgcgttcatcgaacagcgcggcgtcacatcggcctatctcgtc
HisValArgTyrLeuAspAlaPheIleGluGlnArgGlyValThrSerAlaTyrLeuVal
                310 320 330 340 350 360
....|....|....|....|....|....|....|....|....|....|....|....|
gcgcaggactggggcacggcgctcgcatttcatctcgccgcgcgccggccggatttcgta
AlaGlnAspTrpGlyThrAlaLeuAlaPheHisLeuAlaAlaArgArgProAspPheVal
                370 380 390 400 410 420
....|....|....|....|....|....|....|....|....|....|....|....|
cgcggattagccttcatggaattcatccgcccgatgccgacctggcaggatttccaccat
ArgGlyLeuAlaPheMetGluPheIleArgProMetProThrTrpGlnAspPheHisHis
                430 440 450 460 470 480
....|....|....|....|....|....|....|....|....|....|....|....|
accgaggtcgcggaggagcaagatcatgccgaggcggcgagggcggtctttcgcaagttc
ThrGluValAlaGluGluGlnAspHisAlaGluAlaAlaArgAlaValPheArgLysPhe
                490 500 510 520 530 540
....|....|....|....|....|....|....|....|....|....|....|....|
aggacgccgggcgagggtgaggccatgatcctcgaggcgaatgcgttcgtcgagcgcgtt
ArgThrProGlyGluGlyGluAlaMetIleLeuGluAlaAsnAlaPheValGluArgVal
                550 560 570 580 590 600
....|....|....|....|....|....|....|....|....|....|....|....|
ctgcccggcggaatcgtccgcaagctcggcgacgaagaaatggcgccctatcgcacgccg
LeuProGlyGlyIleValArgLysLeuGlyAspGluGluMetAlaProTyrArgThrPro
                610 620 630 640 650 660
....|....|....|....|....|....|....|....|....|....|....|....|
ttcccgacgcccgagagtcgccgccccgttcttgcgtttccccgcgagctgccgatcgca
PheProThrProGluSerArgArgProValLeuAlaPheProArgGluLeuProIleAla
                670 680 690 700 710 720
....|....|....|....|....|....|....|....|....|....|....|....|
ggtgagcctgccgatgtctatgaggcgctccaatccgcccatgcggcgctggccgcatct
GlyGluProAlaAspValTyrGluAlaLeuGlnSerAlaHisAlaAlaLeuAlaAlaSer
                730 740 750 760 770 780
....|....|....|....|....|....|....|....|....|....|....|....|
tcctatccgaaactgctgttcacgggcgaaccgggcgcgctcgtctcgccggaatttgcc
SerTyrProLysLeuLeuPheThrGlyGluProGlyAlaLeuValSerProGluPheAla
                790 800 810 820 830 840
....|....|....|....|....|....|....|....|....|....|....|....|
gagcggtttgcggcctcgctgacgcgttgcgcgttgatccggctcggcgcgggattgcac
GluArgPheAlaAlaSerLeuThrArgCysAlaLeuIleArgLeuGlyAlaGlyLeuHis
                850 860 870 880 890 900
....|....|....|....|....|....|....|....|....|....|....|....|
tatctgcaggaggaccacgctgacgcaatcggccgatcggtggccggctggatcgccggc
TyrLeuGlnGluAspHisAlaAspAlaIleGlyArgSerValAlaGlyTrpIleAlaGly
                910 920 930
....|....|....|....|....|....|...
atcgaagcggtgcgtccgcagctcgccgcgtga
IleGluAlaValArgProGlnLeuAlaAlaEnd
(2) INFORMATION FOR SEQ ID NO: 2:
        (i) SEQUENCE CHARACTERISTICS:
                (A) LENGTH: 891 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 2 (LinB)
                  10 20 30 40 50 60
....|....|....|....|....|....|....|....|....|....|....|....|
atgagcctcggcgcaaagccatttggcgagaagaaattcattgagatcaagggccggcgc
MetSerLeuGlyAlaLysProPheGlyGluLysLysPheIleGluIleLysGlyArgArg
                  70 80 90 100 110 120
....|....|....|....|....|....|....|....|....|....|....|....|
atggcctatatcgatgaagggaccggcgatccgatcctcttccagcacggcaatccgacg
MetAlaTyrIleAspGluGlyThrGlyAspProIleLeuPheGlnHisGlyAsnProThr
                130 140 150 160 170 180
....|....|....|....|....|....|....|....|....|....|....|....|
tcgtcctatctgtggcgcaatatcatgccgcattgcgccgggctgggacggctgatcgcc
SerSerTyrLeuTrpArgAsnIleMetProHisCysAlaGlyLeuGlyArgLeuIleAla
                190 200 210 220 230 240
....|....|....|....|....|....|....|....|....|....|....|....|
tgtgacctgatcggcatgggcgattcggacaagctcgatccgtcggggcccgagcgttat
CysAspLeuIleGlyMetGlyAspSerAspLysLeuAspProSerGlyProGluArgTyr
                250 260 270 280 290 300
....|....|....|....|....|....|....|....|....|....|....|....|
gcctatgccgagcatcgtgactatctcgacgcgctgtgggaggcgctcgatctcggggac
AlaTyrAlaGluHisArgAspTyrLeuAspAlaLeuTrpGluAlaLeuAspLeuGlyAsp
                310 320 330 340 350 360
....|....|....|....|....|....|....|....|....|....|....|....|
agggttgttctggtcgtgcatgactgggggtccgccctcggcttcgactgggcccgccgc
ArgValValLeuValValHisAspTrpGlySerAlaLeuGlyPheAspTrpAlaArgArg
                370 380 390 400 410 420
....|....|....|....|....|....|....|....|....|....|....|....|
caccgcgagcgtgtacaggggattgcctatatggaagcgatcgccatgccgatcgaatgg
HisArgGluArgValGlnGlyIleAlaTyrMetGluAlaIleAlaMetProIleGluTrp
                430 440 450 460 470 480
....|....|....|....|....|....|....|....|....|....|....|....|
gcggattttcccgaacaggatcgcgatctgtttcaggcctttcgctcgcaggcgggcgaa
AlaAspPheProGluGlnAspArgAspLeuPheGlnAlaPheArgSerGlnAlaGlyGlu
                490 500 510 520 530 540
....|....|....|....|....|....|....|....|....|....|....|....|
gaattggtgttgcaggacaatgtttttgtcgaacaagttctccccggattgatcctgcgc
GluLeuValLeuGlnAspAsnValPheValGluGlnValLeuProGlyLeuIleLeuArg
                550 560 570 580 590 600
....|....|....|....|....|....|....|....|....|....|....|....|
cccttaagcgaagcggagatggccgcctatcgcgagcccttcctcgccgccgggaagcc
ProLeuSerGluAlaGluMetAlaAlaTyrArgGluProPheLeuAlaAlaGlyGluAla
                610 620 630 640 650 660
....|....|....|....|....|....|....|....|....|....|....|....|
cgtcgaccgaccctgtcttggcctcgccaaatcccgatcgcaggcaccccggccgacgtg
ArgArgProThrLeuSerTrpProArgGlnIleProIleAlaGlyThrProAlaAspVal
                670 680 690 700 710 720
....|....|....|....|....|....|....|....|....|....|....|....|
gtcgcgatcgcccgggactatgccggctggctcagcgaaagcccgattccgaaactcttc
ValAlaIleAlaArgAspTyrAlaGlyTrpLeuSerGluSerProIleProLysLeuPhe
                730 740 750 760 770 780
....|....|....|....|....|....|....|....|....|....|....|....|
atcaacgccgagccgggagccctgaccacgggccgaatgcgcgacttctgccgcacatgg
IleAsnAlaGluProGlyAlaLeuThrThrGlyArgMetArgAspPheCysArgThrTrp
                790 800 810 820 830 840
....|....|....|....|....|....|....|....|....|....|....|....|
ccaaaccagaccgaaatcacggtcgcaggcgcccatttcatccaggaggacagtccggac
ProAsnGlnThrGluIleThrValAlaGlyAlaHisPheIleGlnGluAspSerProAsp
                850 860 870 880 890
....|....|....|....|....|....|....|....|....|....|.
gagattggcgcggcgattgcggcgtttgtccggcgattgcgcccagcataa
GluIleGlyAlaAlaIleAlaAlaPheValArgArgLeuArgProAlaEnd
(2) INFORMATION FOR SEQ ID NO: 3:
        (i) SEQUENCE CHARACTERISTICS:
                (A) LENGTH: 882 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 3 (DhaA)
                10 20 30 40 50 60
....|....|....|....|....|....|....|....|....|....|....|....|
atgtcagaaatcggtacaggcttccccttcgacccccattatgtggaagtcctgggcgag
MetSerGluIleGlyThrGlyPheProPheAspProHisTyrValGluValLeuGlyGlu
                  70 80 90 100 110 120
....|....|....|....|....|....|....|....|....|....|....|....|
cgtatgcactacgtcgatgttggaccgcgggatggcacgcctgtgctgttcctgcacggt
ArgMetHisTyrValAspValGlyProArgAspGlyThrProValLeuPheLeuHisGly
                130 140 150 160 170 180
....|....|....|....|....|....|....|....|....|....|....|....|
aacccgacctcgtcctacctgtggcgcaacatcatcccgcatgtagcaccgagtcatcgg
AsnProThrSerSerTyrLeuTrpArgAsnIleIleProHisValAlaProSerHisArg
                190 200 210 220 230 240
....|....|....|....|....|....|....|....|....|....|....|....|
tgcattgctccagacctgatcgggatgggaaaatcggacaaaccagacctcgattatttc
CysIleAlaProAspLeuIleGlyMetGlyLysSerAspLysProAspLeuAspTyrPhe
                250 260 270 280 290 300
....|....|....|....|....|....|....|....|....|....|....|....|
ttcgacgaccacgtccgctacctcgatgccttcatcgaagccttgggtttggaagaggtc
PheAspAspHisValArgTyrLeuAspAlaPheIleGluAlaLeuGlyLeuGluGluVal
                310 320 330 340 350 360
....|....|....|....|....|....|....|....|....|....|....|....|
gtcctggtcatccacgactggggctcagctctcggattccactgggccaagcgcaatccg
ValLeuValIleHisAspTrpGlySerAlaLeuGlyPheHisTrpAlaLysArgAsnPro
                370 380 390 400 410 420
....|....|....|....|....|....|....|....|....|....|....|....|
gaacgggtcaaaggtattgcatgtatggaattcatccggcctatcccgacgtgggacgaa
GluArgValLysGlyIleAlaCysMetGluPheIleArgProIleProThrTrpAspGlu
                430 440 450 460 470 480
....|....|....|....|....|....|....|....|....|....|....|....|
tggccggaattcgcccgtgagaccttccaggccttccggaccgccgacgtcggccgagag
TrpProGluPheAlaArgGluThrPheGlnAlaPheArgThrAlaAspValGlyArgGlu
                490 500 510 520 530 540
....|....|....|....|....|....|....|....|....|....|....|....|
ttgatcatcgatcagaacgctttcatcgagggtgcgctcccgaaatgcgtcgtccgtccg
LeuIleIleAspGlnAsnAlaPheIleGluGlyAlaLeuProLysCysValValArgPro
                550 560 570 580 590 600
....|....|....|....|....|....|....|....|....|....|....|....|
cttacggaggtcgagatggaccactatcgcgagcccttcctcaagcctgttgaccgagag
LeuThrGluValGluMetAspHisTyrArgGluProPheLeuLysProValAspArgGlu
                610 620 630 640 650 660
....|....|....|....|....|....|....|....|....|....|....|....|
ccactgtggcgattccccaacgagctgcccatcgccggtgagcccgcgaacatcgtcgcg
ProLeuTrpArgPheProAsnGluLeuProIleAlaGlyGluProAlaAsnIleValAla
                670 680 690 700 710 720
....|....|....|....|....|....|....|....|....|....|....|....|
ctcgtcgaggcatacatgaactggctgcaccagtcacctgtcccgaagttgttgttctgg
LeuValGluAlaTyrMetAsnTrpLeuHisGlnSerProValProLysLeuLeuPheTrp
                730 740 750 760 770 780
....|....|....|....|....|....|....|....|....|....|....|....|
ggcacacccggcgtactgatccccccggccgaagccgcgagacttgccgaaagcctcccc
GlyThrProGlyValLeuIleProProAlaGluAlaAlaArgLeuAlaGluSerLeuPro
                790 800 810 820 830 840
....|....|....|....|....|....|....|....|....|....|....|....|
aactgcaagacagtggacatcggcccgggattgcactacctccaggaagacaacccggac
AsnCysLysThrValAspIleGlyProGlyLeuHisTyrLeuGlnGluAspAsnProAsp
                850 860 870 880
....|....|....|....|....|....|....|....|..
cttatcggcagtgagatcgcgcgctggctccccgcactctag
LeuIleGlySerGluIleAlaArgTrpLeuProAlaLeuEnd
2) INFORMATION FOR SEQ ID NO: 4:
        (i) SEQUENCE CHARACTERISTICS:
                (A) LENGTH: 903 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 4 (DmbA)
                  10 20 30 40 50 60
....|....|....|....|....|....|....|....|....|....|....|....|
atgacagcattcggcgtcgagccctacgggcagccgaagtacctagaaatcgccgggaag
MetThrAlaPheGlyValGluProTyrGlyGlnProLysTyrLeuGluIleAlaGlyLys
                  70 80 90 100 110 120
....|....|....|....|....|....|....|....|....|....|....|....|
cgcatggcgtatatcgacgaaggcaagggtgacgccatcgtctttcagcacggcaacccc
ArgMetAlaTyrIleAspGluGlyLysGlyAspAlaIleValPheGlnHisGlyAsnPro
                130 140 150 160 170 180
....|....|....|....|....|....|....|....|....|....|....|....|
acgtcgtcttacttgtggcgcaacatcatgccgcacttggaagggctgggccggctggtg
ThrSerSerTyrLeuTrpArgAsnIleMetProHisLeuGluGlyLeuGlyArgLeuVal
                190 200 210 220 230 240
....|....|....|....|....|....|....|....|....|....|....|....|
gcctgcgatctgatcgggatgggcgcgtcggacaagctcagcccatcgggacccgaccgc
AlaCysAspLeuIleGlyMetGlyAlaSerAspLysLeuSerProSerGlyProAspArg
                250 260 270 280 290 300
....|....|....|....|....|....|....|....|....|....|....|....|
tatagctatggcgagcaacgagactttttgttcgcgctctgggatgcgctcgacctcggc
TyrSerTyrGlyGluGlnArgAspPheLeuPheAlaLeuTrpAspAlaLeuAspLeuGly
                310 320 330 340 350 360
....|....|....|....|....|....|....|....|....|....|....|....|
gaccacgtggtactggtgctgcacgactggggctcggcgctcggcttcgactgggctaac
AspHisValValLeuValLeuHisAspTrpGlySerAlaLeuGlyPheAspTrpAlaAsn
                370 380 390 400 410 420
....|....|....|....|....|....|....|....|....|....|....|....|
cagcatcgcgaccgagtgcaggggatcgcgttcatggaagcgatcgtcaccccgatgacg
GlnHisArgAspArgValGlnGlyIleAlaPheMetGluAlaIleValThrProMetThr
                430 440 450 460 470 480
....|....|....|....|....|....|....|....|....|....|....|....|
tgggcggactggccgccggccgtgcggggtgtgttccagggtttccgatcgcctcaaggc
TrpAlaAspTrpProProAlaValArgGlyValPheGlnGlyPheArgSerProGlnGly
                490 500 510 520 530 540
....|....|....|....|....|....|....|....|....|....|....|....|
gagccaatggcgttggagcacaacatctttgtcgaacgggtgctgcccggggcgatcctg
GluProMetAlaLeuGluHisAsnIlePheValGluArgValLeuProGlyAlaIleLeu
                550 560 570 580 590 600
....|....|....|....|....|....|....|....|....|....|....|....|
cgacagctcagcgacgaggaaatgaaccactatcggcggccattcgtgaacggcggcgag
ArgGlnLeuSerAspGluGluMetAsnHisTyrArgArgProPheValAsnGlyGlyGlu
                610 620 630 640 650 660
....|....|....|....|....|....|....|....|....|....|....|....|
gaccgtcgccccacgttgtcgtggccacgaaaccttccaatcgacggtgagcccgccgag
AspArgArgProThrLeuSerTrpProArgAsnLeuProIleAspGlyGluProAlaGlu
                670 680 690 700 710 720
....|....|....|....|....|....|....|....|....|....|....|....|
gtcgtcgcgttggtcaacgagtaccggagctggctcgaggaaaccgacatgccgaaactg
ValValAlaLeuValAsnGluTyrArgSerTrpLeuGluGluThrAspMetProLysLeu
                730 740 750 760 770 780
....|....|....|....|....|....|....|....|....|....|....|....|
ttcatcaacgccgagcccggcgcgatcatcaccggccgcatccgtgactatgtcaggagc
PheIleAsnAlaGluProGlyAlaIleIleThrGlyArgIleArgAspTyrValArgSer
                790 800 810 820 830 840
....|....|....|....|....|....|....|....|....|....|....|....|
tggcccaaccagaccgaaatcacagtgcccggcgtgcatttcgttcaggaggacagccca
TrpProAsnGlnThrGluIleThrValProGlyValHisPheValGlnGluAspSerPro
                850 860 870 880 890 900
....|....|....|....|....|....|....|....|....|....|....|....|
gaggaaatcggtgcggccatagcacagttcgtccggcagctccggtcggcggccggcgtc
GluGluIleGlyAlaAlaIleAlaGlnPheValArgGlnLeuArgSerAlaAlaGlyVal
...
tga
End
(2) INFORMATION FOR SEQ ID NO: 5:
        (i) SEQUENCE CHARACTERISTICS:
                (A) LENGTH: 903 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 5 (DmbB)
                  10 20 30 40 50 60
....|....|....|....|....|....|....|....|....|....|....|....|
atggatgtcctacgcaccccagactcccggttcgaacacctggtgggctacccgtttgca
MetAspValLeuArgThrProAspSerArgPheGluHisLeuValGlyTyrProPheAla
                  70 80 90 100 110 120
....|....|....|....|....|....|....|....|....|....|....|....|
ccgcactatgtcgatgtgacggccggcgacacccagccgttgcgaatgcactacgtcgac
ProHisTyrValAspValThrAlaGlyAspThrGlnProLeuArgMetHisTyrValAsp
                130 140 150 160 170 180
....|....|....|....|....|....|....|....|....|....|....|....|
gagggcccgggcgacggtccgccgatcgtcttgctgcacggcgagcccacctggagttat
GluGlyProGlyAspGlyProProIleValLeuLeuHisGlyGluProThrTrpSerTyr
                190 200 210 220 230 240
....|....|....|....|....|....|....|....|....|....|....|....|
ctgtaccgaaccatgattccgccgctctccgccgccgggcaccgtgtgctcgcgcccgac
LeuTyrArgThrMetIleProProLeuSerAlaAlaGlyHisArgValLeuAlaProAsp
                250 260 270 280 290 300
....|....|....|....|....|....|....|....|....|....|....|....|
ctgatcggcttcggccgctccgacaagccgactcgcatcgaggactacacctacctgcgg
LeuIleGlyPheGlyArgSerAspLysProThrArgIleGluAspTyrThrTyrLeuArg
                310 320 330 340 350 360
....|....|....|....|....|....|....|....|....|....|....|....|
cacgtcgagtgggtgacgtcctggttcgagaatctcgacctgcacgacgttacgctcttc
HisValGluTrpValThrSerTrpPheGluAsnLeuAspLeuHisAspValThrLeuPhe
                370 380 390 400 410 420
....|....|....|....|....|....|....|....|....|....|....|....|
gtgcaggactgggggtcattgatcggtctgcgcatcgctgccgagcacggtgaccggatc
ValGlnAspTrpGlySerLeuIleGlyLeuArgIleAlaAlaGluHisGlyAspArgIle
                430 440 450 460 470 480
....|....|....|....|....|....|....|....|....|....|....|....|
gcgcggctggtggtcgccaacgggtttctccccgccgcgcaggggcgcaccccactcccc
AlaArgLeuValValAlaAsnGlyPheLeuProAlaAlaGlnGlyArgThrProLeuPro
                490 500 510 520 530 540
....|....|....|....|....|....|....|....|....|....|....|....|
ttctacgtgtggcgggcgtttgcgcgctattctccggtgcttcccgctggccgtctggtg
PheTyrValTrpArgAlaPheAlaArgTyrSerProValLeuProAlaGlyArgLeuVal
                550 560 570 580 590 600
....|....|....|....|....|....|....|....|....|....|....|....|
aacttcggcaccgtccacagggttcccgccggggtccgagccggctacgatgcacctttc
AsnPheGlyThrValHisArgValProAlaGlyValArgAlaGlyTyrAspAlaProPhe
                610 620 630 640 650 660
....|....|....|....|....|....|....|....|....|....|....|....|
cccgacaaaacgtatcaagccggcgcccgggcgttcccacggttggtgccgacctcaccc
ProAspLysThrTyrGlnAlaGlyAlaArgAlaPheProArgLeuValProThrSerPro
                670 680 690 700 710 720
....|....|....|....|....|....|....|....|....|....|....|....|
gacgatccggcggtaccggccaaccgcgcggcatgggaagccctgggccggtgggacaaa
AspAspProAlaValProAlaAsnArgAlaAlaTrpGluAlaLeuGlyArgTrpAspLys
                730 740 750 760 770 780
....|....|....|....|....|....|....|....|....|....|....|....|
ccgttccttgccatcttcggttatcgcgacccgatactcgggcaagcggacggtccgctg
ProPheLeuAlaIlePheGlyTyrArgAspProIleLeuGlyGlnAlaAspGlyProLeu
                790 800 810 820 830 840
....|....|....|....|....|....|....|....|....|....|....|....|
atcaagcacattcccggcgcggcgggtcagccgcacgcccgcatcaaggccagccacttc
IleLysHisIleProGlyAlaAlaGlyGlnProHisAlaArgIleLysAlaSerHisPhe
                850 860 870 880 890 900
....|....|....|....|....|....|....|....|....|....|....|....|
atccaggaggacagcggaaccgaactcgccgaacgcatgctctcctggcagcaggcaacg
IleGlnGluAspSerGlyThrGluLeuAlaGluArgMetLeuSerTrpGlnGlnAlaThr
...
taa
End
(2) INFORMATION FOR SEQ ID NO: 6:
        (i) SEQUENCE CHARACTERISTICS:
                (A) LENGTH: 861 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 6 (DmbC)
                  10 20 30 40 50 60
....|....|....|....|....|....|....|....|....|....|....|....|
atgagcatcgatttcacgccggatccgcagctgtacccgttcgagtcgcgctggttcgac
MetSerIleAspPheThrProAspProGlnLeuTyrProPheGluSerArgTrpPheAsp
                  70 80 90 100 110 120
....|....|....|....|....|....|....|....|....|....|....|....|
agctcgcgtggacgcatccactacgtcgacgagggcacgggtccgccgatcctgttgtgt
SerSerArgGlyArgIleHisTyrValAspGluGlyThrGlyProProIleLeuLeuCys
                130 140 150 160 170 180
....|....|....|....|....|....|....|....|....|....|....|....|
cacggcaacccgacgtggagtttcctgtatcgggacatcatcgtcgcactgcgggaccgt
HisGlyAsnProThrTrpSerPheLeuTyrArgAspIleIleValAlaLeuArgAspArg
                190 200 210 220 230 240
....|....|....|....|....|....|....|....|....|....|....|....|
ttccgttgtgtggctccggattatctgggtttcgggttatcggagcgtccctcgggattc
PheArgCysValAlaProAspTyrLeuGlyPheGlyLeuSerGluArgProSerGlyPhe
                250 260 270 280 290 300
....|....|....|....|....|....|....|....|....|....|....|....|
gggtaccagatcgacgagcacgcgcgggtgatcggcgaattcgtcgatcacctgggcctg
GlyTyrGlnIleAspGluHisAlaArgValIleGlyGluPheValAspHisLeuGlyLeu
                310 320 330 340 350 360
....|....|....|....|....|....|....|....|....|....|....|....|
gaccgctacctgagcatgggtcaggactggggtggcccgatcagcatggcggtcgctgtc
AspArgTyrLeuSerMetGlyGlnAspTrpGlyGlyProIleSerMetAlaValAlaVal
                370 380 390 400 410 420
....|....|....|....|....|....|....|....|....|....|....|....|
gagcgtgccgaccgggtccgcggcgtcgtgttgggcaacacgtggttctggccggcggac
GluArgAlaAspArgValArgGlyValValLeuGlyAsnThrTrpPheTrpProAlaAsp
                430 440 450 460 470 480
....|....|....|....|....|....|....|....|....|....|....|....|
acgctggcgatgaaggccttcagcagggtgatgtccagcccgccagtgcagtacgcgatc
ThrLeuAlaMetLysAlaPheSerArgValMetSerSerProProValGlnTyrAlaIle
                490 500 510 520 530 540
....|....|....|....|....|....|....|....|....|....|....|....|
ttacggcgcaacttctttgtcgagcgcttgatacccgcgggaaccgagcaccggccgagt
LeuArgArgAsnPhePheValGluArgLeuIleProAlaGlyThrGluHisArgProSer
                550 560 570 580 590 600
....|....|....|....|....|....|....|....|....|....|....|....|
agcgcggtgatggcgcactaccgggcggtgcagcccaacgccgcggcacgccgaggcgta
SerAlaValMetAlaHisTyrArgAlaValGlnProAsnAlaAlaAlaArgArgGlyVal
                610 620 630 640 650 660
....|....|....|....|....|....|....|....|....|....|....|....|
gccgagatgcccaaacagatcctggccgcccgtcccctgctggcacggctcgcccgggag
AlaGluMetProLysGlnIleLeuAlaAlaArgProLeuLeuAlaArgLeuAlaArgGlu
                670 680 690 700 710 720
....|....|....|....|....|....|....|....|....|....|....|....|
gtgccagccacgctgggcaccaagcccaccctgttgatttgggggatgaaggatgtcgca
ValProAlaThrLeuGlyThrLysProThrLeuLeuIleTrpGlyMetLysAspValAla
                730 740 750 760 770 780
....|....|....|....|....|....|....|....|....|....|....|....|
ttcaggccgaaaacgattatccccagactgagtgcgacatttcccgaccacgtcctggtg
PheArgProLysThrIleIleProArgLeuSerAlaThrPheProAspHisValLeuVal
                790 800 810 820 830 840
....|....|....|....|....|....|....|....|....|....|....|....|
gagctgcccaacgccaaacacttcatccaggaggacgcccccgaccggatcgccgccgcg
GluLeuProAsnAlaLysHisPheIleGlnGluAspAlaProAspArgIleAlaAlaAla
                850 860
....|....|....|....|.
atcattgagcgcttcggctga
IleIleGluArgPheGlyEnd
(2) INFORMATION FOR SEQ ID NO: 7:
        (i) SEQUENCE CHARACTERISTICS:
                (A) LENGTH: 987 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 7 (DrbA)
                10 20 30 40 50 60
....|....|....|....|....|....|....|....|....|....|....|....|
atgagttgccgcctctcgtcaaatcgccgcggatcgtcgaaactagccgccatgacgaat
MetSerCysArgLeuSerSerAsnArgArgGlySerSerLysLeuAlaAlaMetThrAsn
                  70 80 90 100 110 120
....|....|....|....|....|....|....|....|....|....|....|....|
cttgctagcgatctgtttccccacccgtcgtcggaattgtccatcgacggtcacacgctg
LeuAlaSerAspLeuPheProHisProSerSerGluLeuSerIleAspGlyHisThrLeu
                130 140 150 160 170 180
....|....|....|....|....|....|....|....|....|....|....|....|
cgctacatcgatacggcggccagctctgacatcccgagttccgcggtcggatcctccgat
ArgTyrIleAspThrAlaAlaSerSerAspIleProSerSerAlaValGlySerSerAsp
                190 200 210 220 230 240
....|....|....|....|....|....|....|....|....|....|....|....|
ggcgagccaacgtttctttgtgtgcatggcaatccgacgtggagcttttactaccggcga
GlyGluProThrPheLeuCysValHisGlyAsnProThrTrpSerPheTyrTyrArgArg
                250 260 270 280 290 300
....|....|....|....|....|....|....|....|....|....|....|....|
atcatcgagcggtatggcaagcagcaacgagtgatcgcggtcgatcacatcggttgtggt
IleIleGluArgTyrGlyLysGlnGlnArgValIleAlaValAspHisIleGlyCysGly
                310 320 330 340 350 360
....|....|....|....|....|....|....|....|....|....|....|....|
cgcagcgacaaaccatcggaagacgaattcccgtacacgatggccgcgcatcgagacaac
ArgSerAspLysProSerGluAspGluPheProTyrThrMetAlaAlaHisArgAspAsn
                370 380 390 400 410 420
....|....|....|....|....|....|....|....|....|....|....|....|
ctgattcggttggtcgacgagttggatctgaagaacgtgatcctgatcgctcacgattgg
LeuIleArgLeuValAspGluLeuAspLeuLysAsnValIleLeuIleAlaHisAspTrp
                430 440 450 460 470 480
....|....|....|....|....|....|....|....|....|....|....|....|
ggtggtgcgattggtttgtcagccatgcatgctcgccgagaccgcttggctgggattggg
GlyGlyAlaIleGlyLeuSerAlaMetHisAlaArgArgAspArgLeuAlaGlyIleGly
                490 500 510 520 530 540
....|....|....|....|....|....|....|....|....|....|....|....|
ttgctgaacacggctgcgttcccaccgccgtacatgcctcagcgaattgccgcgtgccgg
LeuLeuAsnThrAlaAlaPheProProProTyrMetProGlnArgIleAlaAlaCysArg
                550 560 570 580 590 600
....|....|....|....|....|....|....|....|....|....|....|....|
atgccggtgttgggaactcccgcagttcgcggattgaacttgttcgcacgggccgcggtc
MetProValLeuGlyThrProAlaValArgGlyLeuAsnLeuPheAlaArgAlaAlaVal
                610 620 630 640 650 660
....|....|....|....|....|....|....|....|....|....|....|....|
accatggccatgtcgcgtacgaagatgaaacccgatgtcgcagcgggattgctggctccc
ThrMetAlaMetSerArgThrLysMetLysProAspValAlaAlaGlyLeuLeuAlaPro
                670 680 690 700 710 720
....|....|....|....|....|....|....|....|....|....|....|....|
tatgacaattggaagaaccgagtcgcaatcgatcggttcgttcgcgacattcctttgaat
TyrAspAsnTrpLysAsnArgValAlaIleAspArgPheValArgAspIleProLeuAsn
                730 740 750 760 770 780
....|....|....|....|....|....|....|....|....|....|....|....|
gattcgcatcccacgatgaagactcttcggcagctggagtccgatctgccggacctggca
AspSerHisProThrMetLysThrLeuArgGlnLeuGluSerAspLeuProAspLeuAla
                790 800 810 820 830 840
....|....|....|....|....|....|....|....|....|....|....|....|
tcgctacccatctctttgatttggggaatgaaggattggtgttttcgaccggaatgtctg
SerLeuProIleSerLeuIleTrpGlyMetLysAspTrpCysPheArgProGluCysLeu
                850 860 870 880 890 900
....|....|....|....|....|....|....|....|....|....|....|....|
cgacgtttccaatccgtttggcccgacgcggaagtcacggaactggcgacgaccggtcac
ArgArgPheGlnSerValTrpProAspAlaGluValThrGluLeuAlaThrThrGlyHis
                910 920 930 940 950 960
....|....|....|....|....|....|....|....|....|....|....|....|
tatgtgatcgaagactcgcccgaagaaaccttggccgcgattgattcattgctcgcccgc
TyrValIleGluAspSerProGluGluThrLeuAlaAlaIleAspSerLeuLeuAlaArg
                970 980
....|....|....|....|....|..
gtcaaggaacgcatcggtgcggcgtga
ValLysGluArgIleGlyAlaAlaEnd
(2) INFORMATION FOR SEQ ID NO: 8:
        (i) SEQUENCE CHARACTERISTICS:
                (A) LENGTH: 906 base pairs
                (B) TYPE: nucleic acid
                (C) STRANDEDNESS: unknown
                (D) TOPOLOGY: unknown
      (ii) MOLECULE TYPE: DNA (genomic)
    (iii) HYPOTHETICAL: NO
      (xi) SEQUENCE DESCRIPTION: SEQ ID NO: 8 (DhmA)
                  10 20 30 40 50 60
....|....|....|....|....|....|....|....|....|....|....|....|
atgcatgtgctgcgaaccccggactcccgattcgaaaacctggaggactacccgttcgtg
MetHisValLeuArgThrProAspSerArgPheGluAsnLeuGluAspTyrProPheVal
                  70 80 90 100 110 120
....|....|....|....|....|....|....|....|....|....|....|....|
gcgcattatctcgacgtcaccgcgcgcgacacccggccgcttcgcatgcactacctggac
AlaHisTyrLeuAspValThrAlaArgAspThrArgProLeuArgMetHisTyrLeuAsp
                130 140 150 160 170 180
....|....|....|....|....|....|....|....|....|....|....|....|
gaggggccgatcgacgggccaccgatcgtgctgctgcacggcgagcccacctggagctac
GluGlyProIleAspGlyProProIleValLeuLeuHisGlyGluProThrTrpSerTyr
                190 200 210 220 230 240
....|....|....|....|....|....|....|....|....|....|....|....|
ctgtaccgcaccatgatcacgccgctgaccgacgccggaaaccgggtgctggcacccgac
LeuTyrArgThrMetIleThrProLeuThrAspAlaGlyAsnArgValLeuAlaProAsp
                250 260 270 280 290 300
....|....|....|....|....|....|....|....|....|....|....|....|
ttgatcggcttcggccggtcggacaagcccagccggatcgaggactactcctaccagcgg
LeuIleGlyPheGlyArgSerAspLysProSerArgIleGluAspTyrSerTyrGlnArg
                310 320 330 340 350 360
....|....|....|....|....|....|....|....|....|....|....|....|
cacgtggactgggtggtctcctggttcgaacacctcaacctcagcgacgtcacgctgttc
HisValAspTrpValValSerTrpPheGluHisLeuAsnIleSerAspValThrLeuPhe
                370 380 390 400 410 420
....|....|....|....|....|....|....|....|....|....|....|....|
gtgcaggactggggatcattgatcgggctgcgcatcgccgccgagcagcccgaccgggtg
ValGlnAspTrpGlySerLeuIleGlyLeuArgIleAlaAlaGluGlnProAspArgVal
                430 440 450 460 470 480
....|....|....|....|....|....|....|....|....|....|....|....|
ggacggctggtggtggccaacggctttcttcccaccgcgcagcgacgcaccccgcccgcc
GlyArgLeuValValAlaAsnGlyPheLeuProThrAlaGlnArgArgThrProProAla
                490 500 510 520 530 540
....|....|....|....|....|....|....|....|....|....|....|....|
ttctacgcgtggcgagccttcgcgcgctactcccccgtgctgcccgccggccgcatcgtc
PheTyrAlaTrpArgAlaPheAlaArgTyrSerProValLeuProAlaGlyArgIleVal
                550 560 570 580 590 600
....|....|....|....|....|....|....|....|....|....|....|....|
agcgtcgggaccgtccgccgggtttcgtccaaggtgcgtgccggctacgacgcgcccttc
SerValGlyThrValArgArgValSerSerLysValArgAlaGlyTyrAspAlaProPhe
                610 620 630 640 650 660
....|....|....|....|....|....|....|....|....|....|....|....|
cccgacaagacgtatcaggccggggcgcgggcatttccgcaactggtgcccacctcgccg
ProAspLysThrTyrGlnAlaGlyAlaArgAlaPheProGlnLeuValProThrSerPro
                670 680 690 700 710 720
....|....|....|....|....|....|....|....|....|....|....|....|
gccgatcccgcgattccggccaaccgcaaggcgtgggaagccctcggccgctgggaaaaa
AlaAspProAlaIleProAlaAsnArgLysAlaTrpGluAlaLeuGlyArgTrpGluLys
                730 740 750 760 770 780
....|....|....|....|....|....|....|....|....|....|....|....|
ccgttcctggccatcttcggcgcccgcgaccccatcctcggccacgcggacagtccgctg
ProPheLeuAlaIlePheGlyAlaArgAspProIleLeuGlyHisAlaAspSerProLeu
                790 800 810 820 830 840
....|....|....|....|....|....|....|....|....|....|....|....|
atcaagcacattccgggcgccgcgggccaaccgcacgcccgcatcaacgccagtcacttc
IleLysHisIleProGlyAlaAlaGlyGlnProHisAlaArgIleAsnAlaSerHisPhe
                850 860 870 880 890 900
....|....|....|....|....|....|....|....|....|....|....|....|
atccaggaggaccgcggacctgaactggccgaacgcatcctgtcctggcagcaggcgctg
IleGlnGluAspArgGlyProGluLeuAlaGluArgIleLeuSerTrpGlnGlnAlaLeu
....|.
ctctga
LeuEnd
```

### INDUSTRIAL APPLICABILITY

The invention can be applied for production of optically active compounds, particularly halohydrocarbons, haloalcohols, alcohols, halopolyols and polyols using hydrolytic dehalogenation of racemic or prochiral halegenhydrocarbons by dehalohenation catalysed by haloalkane dehalogenases (the enzyme code number EC 3.8.1.5).

1. A method of production of optically active halohydrocarbons and alcohols using hydrolytic dehalogenation catalysed by a haloalkane dehalogenase characterized in that at least one wild type or modified haloalkane dehalogenase selected from the group of the haloalkane dehalogenases (EC 3.8.1.5) or their mixtures is affected by at least one racemic or prochiral chlorinated, brominated or iodinated compound at the temperature ranged between +10 and +70° C. and pH value between 4.0 and 12.0, in aqueous system or in a monophasic organic solution or in a monophasic organic/aqueous solution or in organic/aqueous biphasic systems.

2. The method according to claim 1 characterized in that the haloalkane dehalogenase is at least one wild type or modified haloalkane dehalogenase selected from the group consisting of:
haloalkane dehalogenase DbjA SEQ ID NO: 1,
haloalkane dehalogenase LinB SEQ ID NO: 2,
haloalkane dehalogenase DhaA SEQ ID NO: 3,
haloalkane dehalogenase DmbA SEQ ID NO: 4,
haloalkane dehalogenase DmbB SEQ ID NO: 5,
haloalkane dehalogenase DmbC SEQ ID NO: 6,
haloalkane dehalogenase DrbA SEQ ID NO: 7,
haloalkane dehalogenase DhmA SEQ ID NO: 8.

3. The method according to claim 1 characterized in that the haloalkane dehalogenase is at least one wild type or modified polypeptide with haloalkane dehalogenase activity having an amino acid sequence that corresponds at least in 90% to the sequence SEQ ID NO: 1, 2, 3, 4, 5, 6, 7 or 8.

4. The method according to claim 1 characterized in that the haloalkane dehalogenase is at least one wild type or modified polypeptide with haloalkane dehalogenase activity having the amino acid sequence that corresponds at least in 80% to the sequence SEQ ID NO: 1, 2, 3, 4, 5, 6, 7 or 8.

5. The method according to claim 1 characterized in that it is performed at presence of surfactants to allow using of enhanced reagent concentration.

6. The method according to claim 1 characterized in that the enzyme halolkane dehalogenase is in soluble or crystalline or lyophilized or precipitated form.

7. The method according to claim 1 characterized in that the enzyme haloalkane dehalogenase is immobilized by adsorption or ionic binding or covalent attachment onto the surface of a macroscopic carrier material.

8. The method according to claim 1 characterized in that the enzyme haloalkane dehalogenase is immobilized by cross-linking or confined to a solid matrix or membrane-restricted compartments.
